(19) United States
(12) Patent Application Publication
(10) Pub. No.: US 2011/0020174 A1
(43) Pub. Date: Jan. 27, 2011

(54) APPARATUS AND METHOD FOR TREATING FORMED PARTS BY MEANS OF HIGH-ENERGY ELECTRON BEAMS

(76) Inventors: Johannes Rauschnabel, Fichtenau (DE); Steffen Ebert, Crailsheim (DE); Oliver Ullmann, Satteldorf (DE); Reinhold Schmieg, Wallhausen (DE)

Correspondence Address: RONALD E. GREIGG;GREIGG & GREIGG P.L.L.C., 1423 POWHATAN STREET, UNIT ONE, ALEXANDRIA, VA, 22314, US

(21) Appl. No.: 12/866,574

(22) PCT Filed: Nov. 28, 2008
(86) PCT No.: PCT/EP2008/066413

§ 371 Date: Sep. 17, 2010

(30) Foreign Application Priority Data
Feb. 6, 2008 (DE) 102008007662.7

(51) Int. Cl. A61L 2/00; G21G 5/00

(52) U.S. Cl. 422/22; 250/453.11

(57) ABSTRACT

The invention relates to a device for treating formed parts with high-energy electron beams. The electron beams are guided by two opposite stationary or displaceable electron discharge windows onto the formed part and bounding a process space for the formed part. A transport device is present for the formed part, by which the formed part can be guided through the process space to the electron discharge windows disposed substantially vertically perpendicular to the transport direction. A channel is disposed in the process space for transporting the formed part and largely shielded against the X-ray radiation.

### PRIOR ART

The invention relates to an apparatus and a method for treating formed parts by means of high-energy electron beams, and in particular also for modifying material properties of the surface and in the peripheral region of three-dimensional formed parts as generically defined by the preamble to the apparatus claim and method claim.

From International Patent Disclosure WO 2007/107331 A1, it is already known that a three-dimensional formed part is treated by means of electrons and in the process comes to rest between two electron discharge windows, on at least one of which an electron accelerator is disposed. Reflectors are also disposed here in such a way that particularly in lateral regions of the formed part that might be subjected to electrons inadequately, for instance because of shading, the electrons are directed in particular from the lateral regions onto the formed part.

From German Patent DE 40 28 479 C2, it is also known that for near-surface heat treatment of aluminum parts of an internal combustion engine, high-energy electron beams are used. For that purpose, the surface is subjected to the electron beam in an area-covering point matrix, and the beam then jumps from point to point and remains for a predetermined length of time at each point for the purpose of heat input.

It is thus known per se that by means of electrons, energy can be introduced into materials in a spatially and chronologically determined way in order to modify their material properties at the surface and within predetermined limits in the volume as well. As a rule, the electrons required for this are generated, formed and accelerated in an electron accelerator, before they are guided, in a further conventional arrangement, for example via what is usually a flat electron discharge window out of the high vacuum to a higher pressure level in the processing chamber with the formed part. Usually, a constant electron density over the entire extent of the electron discharge window is desired. After penetration of the gas layer (which is air, for example) in the distance between the electron discharge window and the formed part, the electrons reach the product surface that is to be treated.

As electron accelerators, area beam generators, also called band emitters, or axial beam generators, known per se, are used. A typical electron accelerator embodied as an axial beam generator additionally includes an electron beam deflection chamber with a beam deflection system, by means of which an electron beam that is generated is periodically diverted over the entire electron discharge window and on average over time in all portions of the window with approximately the same dwell time.

Three-dimensional formed parts, such as packages, medical implants, sets of operating room instruments, prostheses of various materials, such as plastic, paper, metal, or ceramic, are used in various branches of industry, such as the packaging industry, pharmacy, medical technology, or the plastic industry. For certain applications, a change of property is necessary, such as sterilization, surface functionalization, or cross-linking or hardening of the entire surface and the peripheral layer of the formed part.

From German Patent Disclosure DE 199 42 142 A1, it is also known to vary properties of the surface of bulk goods by means of electron energy by moving these bulk goods in a plurality of passes and in a varied position past an electron discharge window.

Such apparatuses for generating electrons for modifying formed part properties are designed such that over the entire electron discharge window, approximately the same electron energy density is generated and output. The prior art transporting systems guide the formed parts without rotation or pivoting, always in the same position, through the treatment zone of the electron accelerator. So that the entire formed part surface will be subjected to electron energy, a change in the position of the formed part is often made during one multiple pass. A disadvantage of this is that it involves relatively great expenditure in terms of time and equipment. Changing the position of the formed part between the individual passes can also not be done randomly but instead must be adapted in such a way that individual surface regions are not in total subjected to different electron energy densities, which would lead to different properties.

From the reference “Technische Beschreibung [Technical Description] Electron Beam Surface Sterilisation System 200 KeV—The Ke VAC S” put out by Linac Technologies, it is also known that the entire surface irradiation of a three-dimensional formed part is done in modified form during only a single pass by means of electron energy by disposing a plurality, that is, at least two or three, electron discharge windows in such a way that they surround the cross section of the formed part, and the formed part is passed between these electron discharge windows, and thus the entire three-dimensional surface is subjected to electrons.

An apparatus for sterilizing the surface of formed parts by means of electron energy also known per se in which the electron accelerator is disposed such that its associated electron discharge windows surround a volume having the cross section of an isosceles triangle, through which the formed parts to be sterilized are guided in one pass. With such apparatuses, although the time expenditure is reduced compared to other known embodiments in which a formed part is subjected to electrons in a plurality of passes, the technological effort and expense are nevertheless very high, because three electron accelerators are used.

Arrangements of three electron discharge windows are also known in which the electrons are, however, generated only by means of an electron accelerator and are distributed over the three electron discharge windows with the aid of a deflection system. All of these known embodiments having a plurality of electron discharge windows make use of the advantage that the electron accelerators, as a result of their triangular disposition, do not affect one another, or affect one another only negligibly, which means that the accelerated electrons of one electron accelerator do not output considerable energy components to the other respective electron accelerators. This is necessary in order to limit the proportion of energy absorbed in the electron discharge window and thus to limit its operating temperature to a subcritical amount. If the material use temperature were exceeded, the vulnerable material of the window covering would otherwise be destroyed by the mechanical strain from the externally applied atmospheric pressure in comparison to the high vacuum in the interior of the beam generator. For titanium foil that is typically used in electron discharge windows, a maximum temperature of about 400° C. must in no case be exceeded. For continuous operation, the assumption is a maximum of 200 to 250° C.

One possibility for limiting the temperature of two opposed electron discharge windows that is known from U.S. Pat. No. 2,741,704 A is the disposition of an additional absorber, such as an at least partly transparent conveyor belt, between the electron discharge windows. In that case, a considerable proportion of energy strikes the absorber, which limits the projection of additional energy onto the opposite electron discharge window.

In the known devices in which two or more electron discharge windows surround one formed part and in which, over one complete electron discharge window, approximately the same electron energy density is output, and a formed part is subjected to electrons in only one pass, individual surface portions of the formed part, depending on its geometry and the resultant different spacing of the surface portions from an electron discharge window, can be subjected to a different dose (energy per unit of surface area or energy per unit of mass) of electron energy.

To achieve a certain property of a formed part, a defined dose of electron energy is necessary. Expediently, the power of the electron generator is adjusted such that at those surface regions where the least dose occurs, the dose occurring there corresponds precisely or at least to the dose that is necessary for modifying the property. All the other surface regions of the formed part are necessarily subjected to a higher dose. This higher dose of energy is also called an overdose. The higher the overdose is in individual regions of a formed part, the greater do the properties in those regions differ from the target parameters. A parameter called an overdose factor indicates the multiplier by which a requisite dose for establishing a desired property is exceeded.

With the known devices, depending on the geometry of the formed parts to be treated, in individual surface regions overdose factors are attained that are unacceptable for many applications for achieving adequately uniform properties over the entire surface. For attaining high productivities, an adapted high conveying speed of the formed parts is necessary. Because of the proportionality of the conveying speed and the beam current, attaining a technologically predetermined minimum dose, which for the field of attaining sterilization is 25 kGy, for example, requires increasing the beam current proportional to speed, which leads to a disproportionate increase in the operating temperature of the electron discharge windows.

### DISCLOSURE OF THE INVENTION

The invention is based on an apparatus for treating formed parts with high-energy electron beams, in which the apparatus has two opposed stationary or movable electron discharge windows, which define a processing chamber for the formed part. According to the invention, there is advantageously a transporting device for the formed part, with which device the formed part can be guided through the processing chamber past electron discharge windows disposed essentially vertically and perpendicular to the transporting direction, and for the delivery of the formed part, a conduit that is largely shielded from the X-radiation in the processing chamber is disposed.

Especially advantageously, the conduit for the delivery is angled twice each upstream of the processing chamber and downstream of the processing chamber, such that the resultant labyrinthine offset of the conduit shields the X-radiation from the inlet and the outlet of the apparatus. Preferably, in each angled conduit portion there is a device with which the formed part can be transferred from a horizontal delivery to an inclined position for being transported in the processing chamber and can be returned again afterward.

This device is for example a gripper that is controllable in a plurality of degrees of freedom, which is preferably equipped with an inflatable and/or electromagnetic gripper element. Because of the proposed gripper mechanism and because the formed parts are placed upright, an offset that virtually predetermines the labyrinth required for the shielding is automatically created in the conduit, the conduit being angled for that purpose. The X-radiation generated in the treatment zone of the processing chamber is thus shielded or reflected to the inlet and outlet; this makes a very compact structure with very robust mechanics possible, and makes other, otherwise necessary, complicated constructions for the conduit, tunnel or gate superfluous.

Advantageously, in the conduit, lead-doped glass or plastic windows can be disposed, through which, by means of an optical sensor system, observation and/or control of the formed parts and their transporting can be performed. The optical sensor system can comprise light gates and/or in particular electronic cameras and is thus advantageously located outside the (lead) conduit.

It is also advantageous that an air flow can be directed in the direction of the formed part and past it. For controlling the air flow, foldable or otherwise controllable baffles can be present upstream and downstream in the processing chamber, with which baffles, in predetermined peripheral regions of the formed part, the flow cross sections are variable for controlling the pressure in the conduit.

With the aid of so-called “paddles”, for instance, as controllable baffles in the processing chamber, the air pressure control proposed here in the conduit is advantageous above all because the formed parts to be treated are not a hose but discrete objects with a defined spacing from one another, and the air flowing along the formed parts thus experiences fluctuations at the constrictions and interfaces with the surroundings, and these fluctuations are expressed in pressure fluctuations. To compensate for these fluctuations, the paddle can be used in such a way that the flow cross section is artificially reduced whenever the air could too easily flow out into adjoining regions and thus would lower the pressure.

An advantageous embodiment of the apparatus of the invention is also obtained if, in a manner that is known per se from the prior art mentioned at the outset, in the processing chamber, there is at least one reflector, which serves as a further lateral boundary of the processing chamber and with which electron beams can be directed to the lateral surfaces and/or peripheral layers of the formed part. The electron discharge windows and the transporting device can also be inclined from the horizontal by a predetermined angle.

One advantageous embodiment is obtained with band emitters, disposed in or downstream of the electron discharge windows, which emitters comprise virtually vertically extending wires disposed side by side that act as filaments that emit electrons. Alternatively, however, it would also be possible instead of the band emitters for a scanner with a point emitter to be disposed here.

Because of the vertical position of the electron beam generators, in band emitters the not entirely avoidable sagging of the electron-emitting filament, which as a rule is a tungsten wire, is neutralized for the emissions characteristic. In an emitter that emits from top to bottom, the result, in what is then a horizontal filament that is retained only at the ends, is downward sagging over the course of the service life. This so-called belly has a tendency to bend the electron curtain toward the sides, so that it does not meet the electron discharge window but instead a relevant portion of it goes into the walls and thus into the anode. In emitters that emit upward, which should as a rule be avoided because of the particle load, focusing the electron curtain takes place to a virtual focal point, and thus the sides of the formed part to be treated receive a lesser dose. The two bellies described thus may require readjustment over the course of the service life; this is not necessary in the band emitter proposed according to the invention, in the form of a vertically placed electron beam generator, since the characteristic of the electron curtain does not vary over the course of the service life, and the yield is optimal.

Even in the already known versions with three emitters in a star arrangement, two emitters at a time emit from obliquely below, so that the belly in this case asymmetrically shifts the electron curtain, which also deposits a large amount of electron energy in one wall side, and that energy is not available for treating the formed parts.

In an advantageous method for treating formed parts with high-energy electron beams with one of the apparatuses described above, the formed part can be guided via the conduit into the processing chamber and there, in the stationary state or in the state moved past it, is subjected to electron beams by means of a single or multiple irradiation operation, in which the processing chamber acts as a gate. Preferably, the passage through the system of the formed parts is done in a batch mode; and that the number of formed parts, per unit of time in the passage through the system is independent of the constant passage speed, and as a function of the cycle time and mode of operation, a different insertion of the formed parts by the gripper into the passage system takes place.

In a further advantageous embodiment, the processing chamber opposite the inlet and outlet of the conduit can be partitioned off with double bulkhead gates in both cases, and for passing the formed parts to be treated these gates can be opened in alternation; on each side of the treatment region, one bulkhead must always be closed. In a preferred embodiment, the bulkheads are coated with load, to shield against X-radiation from the processing chamber. In this embodiment, a labyrinth is not necessary, since the radiation is shielded toward the inlet and outlet.

A further advantageous embodiment contains a chamber, partitioned off by a lead-coated bulkhead, in the outlet region, which chamber is capable of holding at least one formed part that can be removed from outside via a further bulkhead or a door. This chamber is ideally disposed perpendicular to the outlet conveyor belt, and with the bulkhead open, the formed part can be pushed over into the chamber by means of a translational motion perpendicular to the outlet direction. After closure of the bulkhead, the formed part can then be removed via the outer door, without having to interrupt the irradiation operation. In an especially advantageous embodiment, the outer door comprises a double-lidded gate, with which sterile removal of the formed part is possible.

An advantageous use of the method or of the apparatus is obtained in a treatment, preferably a surface treatment with high-energy electron beams, is done for modifying plastics, for sterilizing products or intermediate products, especially medical products, for disinfecting and/or sterilizing packages, for hardening coatings, or for disinfecting and/or sterilizing objects or foods.

The invention therefore advantageously solves a series of technical problems and makes it possible to create both an apparatus with a cyclically operating transporting device and a method which overcome the disadvantages of the prior art in treatment with electron beams. In particular, the apparatus and the method are suitable for modifying properties of three-dimensional formed parts, with little expenditure of time or technology, in such a way that a sufficiently uniform modification of the entire surface or of the peripheral regions of the formed parts is attained and nevertheless, no productivity-limiting disadvantages result from the overall arrangement of the electron accelerators.

### BRIEF DESCRIPTION OF THE DRAWING

The invention will be explained below in terms of the exemplary embodiment shown in the drawing FIGURE. In the drawing:

FIG. 1 is a schematic illustration of an apparatus according to the invention for treating formed parts with electron beams, having a processing chamber.

### EMBODIMENT OF THE INVENTION

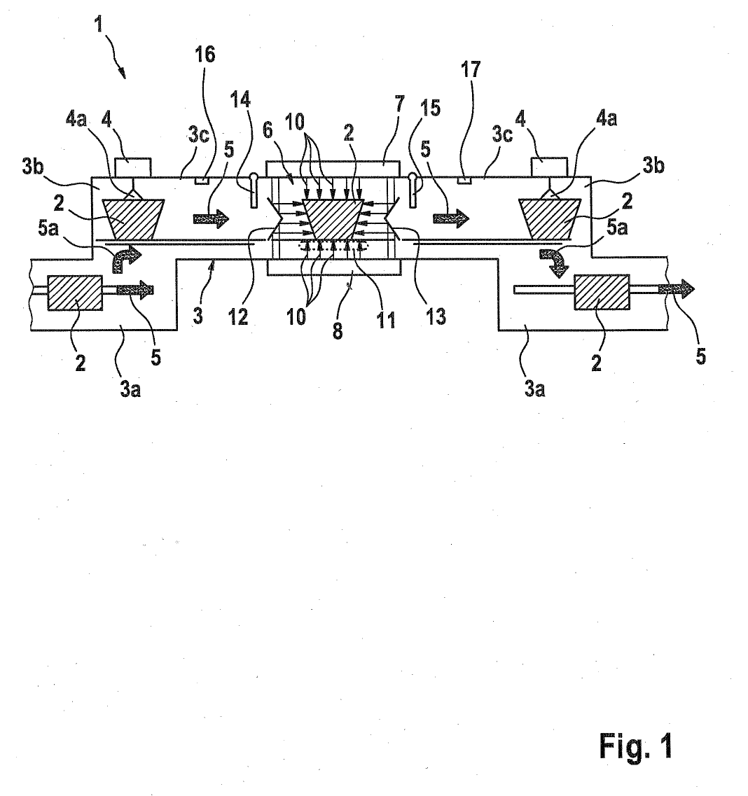

In FIG. 1, an apparatus 1 for electron treatment for the sake of sterilizing the surface of a formed part 2 is shown in a top view on the transporting plane in a conduit 3. The formed part 2 here is a three-dimensional object of rectangular shape in top view, as can be seen from the conduit portions 3a at the inlet and at the outlet.

One gripper 4 is disposed in each conduit portion 3b; under suitable control and having with a gripper element 4a, it puts the formed part 2 into an upright position, so that a trapezoidal cross section of the formed part 2 in the conduit portions 3c can now be seen. Arrows 5 indicate the transporting direction for the formed parts 2, and arrows 5a are intended to symbolically illustrate the rotation from the horizontal to an upright position of the formed part 2.

A processing chamber 6 is present here as a treatment zone for the formed parts 2; it is defined by two parallel, opposed electron discharge windows 7 and 8, extending vertically into the plane of the drawing, each with one band emitter, not described in further detail and known per se from the prior art, for generating the high-energy electron beams 10.

Between the two electron discharge windows 7 and 8, the formed part 2 is passed continuously, or with a treatment stop, on a conveyor belt system 11 through the processing chamber, and in the process, the entire surface of the formed part 2 is subjected to electron beams 10.

At the oblique side faces of the formed part 2 in its trapezoidal cross section, the least energy dose in each case would be transmitted at the points farthest away from the electron discharge windows. Reflectors 12 and 13, for instance of gold, are therefore disposed here as a lateral boundary of the processing chamber 6, in such a way that the electron beams from the respective electron discharge window 7 or 8 are reflected at the inclined faces of the reflectors 12 and 13. The otherwise unused peripheral rays of the electron beams 10 are thus guided, by the angular disposition of the reflectors 12 and 13, into the region that without the reflectors would be the lowest dose of electron beams 10 to the formed part 2.

In the interior of the conduit portion 3c, an overpressure is necessary here, but at the outlets and inlets into and out of the conduit portions 3a, an underpressure should prevail, so that an air flow, to a suction device, for example, takes place counter to the transporting direction. The air flow should therefore be directed in the direction of the formed part 2 and past it. For controlling this air flow in the processing chamber 6, there are flaplike or otherwise controllable baffles 14 and 15 upstream and/or downstream of the formed part 2, and with them, the flow cross sections in predetermined peripheral regions of the formed part 2 are variable for controlling the pressure in the conduit portion 3c.

Lead-doped glass or plastic windows 16 and 17 are also disposed in the conduit portion 3c, and by them, by means of an optical sensor unit available on the market and not shown here, observation and/or control of the formed parts 2 and their transport can be performed. The optical sensor unit may comprise light gates mounted outside the conduit portion 3c or on other conduit portions, and/or it may in particular comprise electronic cameras.

In summary, concrete embodiments of the apparatus, described above as an exemplary embodiment, that functions automatically and in cyclical fashion for irradiating the formed part 2 are described:
As a fundamental structure or housing of the apparatus 1 or of the conduit 3, a welded steel construction of special steel of compact labyrinthine design, with a lead-reinforced protective lining, can be used as X-ray protection, and all the drive mechanisms and the electron accelerators of the band emitters 7 and 8 are disposed on the outside and thus make optimal accessibility possible. All the materials installed in the interior should be designed to be both ozone- and H₂O₂-resistant.
A horizontal inlet belt can be provided for manually depositing or transferring the formed part 2, for instance from a so-called preceding bag opener and for safer and more-precise guidance of the formed part 2 through a lateral guides on a belt.
The gripper 4, as a transfer station, can be a suitable handling unit with an inflatable rubber seal on the gripper element 4a for the sake of form-locking and gentle gripping, hoisting, tilting, and linear displacement for transferring the formed part 2 to passage through the system, and the sealing material is ozone- and H₂O₂-resistant, and automatic pressure monitoring for checking the rubber seal for leaks is optionally done.
In the processing chamber 6, for passage through the system of the formed part 2, a double, structurally separate round belt system may be used as a conveyor belt system 11 for continuously transporting the formed part 2 through the electron accelerator treatment zone, this belt system also comprising two special steel round belts for guiding the formed part 2 at the top and bottom, for introducing the formed part 2 into the electron accelerator treatment zone, and two special steel round belts for guiding the formed part at the top and bottom, for carrying the formed part 2 out of the electron accelerator treatment zone in the processing chamber 6.
The plane of passage through the system in the treatment zone may also be tilted in relation to the passage direction; that is, the formed parts 2 are guided with an inclination of 90°±30° through the electron accelerator treatment zone in the processing chamber. Then, at the direct transfer point between the first and second round belt system, the transport can be done in contactless fashion, as a result of which all the surfaces of the formed part 2 are successively exposed to the electron radiation.
The transport outward of the formed parts 2 is done analogously to the delivery, with a reverse rotation of the formed parts 2.
For irradiating a formed part 2 inside the processing chamber 6 between the two electron discharge windows 7 and 8, various alternative possibilities are available. For instance, a formed part 2 can be guided at constant speed through the processing chamber 6 and during that time subjected to electron beams 10. Alternatively, it is possible for the formed part 2 to be guided into the processing chamber 6 and subjected to electrons there in the stationary state, by means of a single or multiple irradiation operation.

1-15. (canceled)

16. An apparatus for the treatment of three-dimensional formed parts with high-energy electron beams, in which the electron beams are guided by two opposed stationary or movable electron discharge windows which define a processing chamber for one of the three-dimensional formed parts, for subjecting the entire surface area of the three-dimensional formed part to the electron beams, wherein for delivery of the formed part a conduit is disposed so as to be largely shielded from X-radiation in the processing chamber, the conduit for the delivery being angled twice, once upstream of the processing chamber and once downstream of the processing chamber, such that a resultant labyrinthine offset of the conduit shields the X-radiation from an inlet and an outlet of the apparatus, wherein a transporting device for the three-dimensional formed part is present, with which the formed part can be guided through the processing chamber, past the electron discharge windows disposed essentially vertically to the transporting direction, and wherein that in each angled conduit portion, there is a device with which the three-dimensional formed part is transferred from a horizontal delivery to an inclined position for being transported in the processing chamber and is returned again afterward.

17. The apparatus as defined by claim 16, wherein the device is a gripper that is controllable in a plurality of degrees of freedom, which is preferably equipped with an inflatable and/or electromagnetic gripper element.

18. The apparatus as defined by claim 16, wherein in the conduit, lead-doped glass or plastic windows are disposed, through which, by means of an optical sensor system, observation and/or control of the formed parts and their transporting can be performed.

19. The apparatus as defined by claim 17, wherein in the conduit, lead-doped glass or plastic windows are disposed, through which, by means of an optical sensor system, observation and/or control of the formed parts and their transporting can be performed.

20. The apparatus as defined by claim 18, wherein the optical sensor system comprises light gates and/or cameras.

21. The apparatus as defined by claim 19, wherein the optical sensor system comprises light gates and/or cameras.

22. The apparatus as defined by claim 16, wherein an air flow can be directed in a direction of the formed part and past it.

23. The apparatus as defined by claim 21, wherein an air flow can be directed in a direction of the formed part and past it.

24. The apparatus as defined by claim 22, wherein for controlling the air flow, foldable or otherwise controllable baffles are present upstream and downstream in the processing chamber, with which baffles, in predetermined peripheral regions of the formed part, flow cross sections are variable for controlling pressure in the conduit.

25. The apparatus as defined by claim 23, wherein for controlling the air flow, foldable or otherwise controllable baffles are present upstream and downstream in the processing chamber, with which baffles, in predetermined peripheral regions of the formed part, flow cross sections are variable for controlling pressure in the conduit.

26. The apparatus as defined by claim 16, wherein in the processing chamber, there is at least one reflector, which serves as a further lateral boundary of the processing chamber and with which electron beams can be directed to the lateral surfaces and/or peripheral layers of the formed part.

27. The apparatus as defined by claim 25, wherein in the processing chamber, there is at least one reflector, which serves as a further lateral boundary of the processing chamber and with which electron beams can be directed to the lateral surfaces and/or peripheral layers of the formed part.

28. The apparatus as defined by claim 16, wherein downstream of the electron discharge windows, band emitters are disposed, for which virtually disposed wires serve as electron-emitting filaments.

29. The apparatus as defined by claim 27, wherein downstream of the electron discharge windows, band emitters are disposed, for which virtually disposed wires serve as electron-emitting filaments.

30. The apparatus as defined by claim 16, wherein the electron discharge windows and the transporting device are inclined from horizontal by a predetermined angle.

31. The apparatus as defined by claim 16, wherein in an outflow region, a chamber is disposed perpendicular to transport of the formed part and is separated from the outlet conduit by a bulkhead and has an opening in a chamber wall for withdrawing a formed part, which opening is closed with a door or double-lidded gate.

32. A method for treating formed parts with high-energy electron beams, having an apparatus as defined by claim 16, wherein the formed part is guided via the conduit into the processing chamber and there, in a stationary state or in a state moved past it, is subjected to electron beams by means of a single or multiple irradiation operation, in which the processing chamber acts as a gate.

33. The method as defined by claim 32, wherein passage through the system of the formed parts is done in a batch mode, the number of formed parts per unit of time in the passage through the system is independent of a constant passage speed, and as a function of a cycle time and mode of operation, a different insertion of the formed parts by the gripper into the system takes place.

34. The use of an apparatus as defined by claim 16, wherein the treatment, preferably for surface treatment with high-energy electron beams, is done for modifying plastics, for sterilizing products or intermediate products, especially medical products, for disinfecting and/or sterilizing packages, for hardening coatings, or for disinfecting and/or sterilizing objects or foods.

35. The use of a method as defined by claim 32, wherein the treatment, preferably for surface treatment with high-energy electron beams, is done for modifying plastics, for sterilizing products or intermediate products, especially medical products, for disinfecting and/or sterilizing packages, for hardening coatings, or for disinfecting and/or sterilizing objects or foods.
